(19) United States
(12) Patent Application Publication
(10) Pub. No.: US 2004/0220494 A1
(43) Pub. Date: Nov. 4, 2004

(54) Method for objective verification of auditory steady-state responses (ASSR) in the time domain

(76) Inventor: Ekkehard Sturzebecher, Petershagen (DE)

Correspondence Address: DYKEMA GOSSETT PLLC, FRANKLIN SQUARE, THIRD FLOOR WEST, 1300 I STREET, NW, WASHINGTON, DC, 20005, US

(21) Appl. No.: 10/772,426

(22) Filed: Feb. 6, 2004

(30) Foreign Application Priority Data
Feb. 7, 2003 (EP) 03388009.7

(51) Int. Cl. A61B 5/00

(52) U.S. Cl. 600/559; 128/920

(57) ABSTRACT

The invention relates to a method for objective verification of auditory steady-state responses (ASSR) in the time domain whereby the individual epochs are transformed into the frequency domain. The response can be made free from noise of the asynchronous EEG in the frequency domain; the amplitudes of the spectral lines, which contain only noise, are set at zero, and the spectral lines of the harmonics remain uninfluenced. The altered spectrum is subsequently transformed back into the time domain.

The invention relates to the field of objective measuring of hearing ability by using evoked auditory steady-state responses (ASSR). The proposed method for objective verification of ASSR in the time domain can be employed in an ASSR-based hearing screening of a newborn as well as in objective audible threshold measuring based on ASSR.

There are two different types of ASSR known:

1. Click-Evoked ASSR

2. ASSR evoked through a continuous amplitude or frequency-modulated tone, which are also referred to as amplitude-modulation following response (AMFR).

Both types of ASSR are described in the frequency domain by so-called harmonics (one fundamental wave and several harmonic waves). The frequency of the fundamental wave corresponds to the click stimulus rate or the frequency of the modulation signal. The frequencies of the harmonic waves are multiples of the frequency of the fundamental wave. This means that the entire response is represented by a few spectral lines. The substantial portion of the noise power caused by the asynchronous electroencephalogram (asynchronous EEG) is concentrated, in contrast, on the spectral lines lying between the harmonics.

The objective verification of the ASSR occurred heretofore exclusively in the frequency domain. Several statistical methods are known in this regard which interpret as so-called one-sample tests only the phase or also the phase plus the amplitude of one individual spectral line, preferably the first harmonic (fundamental wave) (Stapells D R, Makeig S, Galambos R. Auditory steady-state responses: Threshold prediction using phase coherence. Electgroencephalography and Clinical Neurophysiology 1987;67:260-270; Valdes J L, Perez-Abalo M C, Martin V, Savio G, Sierra C, Rodriguez E, Lins O. Comparison of statistical indicators for the automatic detection of 80 Hz auditory steady state response (AMFR). Ear and Hearing 1997;18:420-429). The recorded time signal is transformed into the frequency domain for this purpose. The length of the transformed epochs must be selected in such a manner that the epoch is an integral multiple of the length of the period of the click stimulus rate or of the modulation frequency. The spectral line (fundamental frequency) corresponding to the click stimulus rate or the modulation frequency is searched and tested in the frequency spectrum that exists after the transformation. The advantage of the response verification in the frequency domain, compared to the direct verification in the time domain, is that the portion of the noise power in the spectral domain (represented by the spectral lines lying between the harmonics) does not interfere with the response detection since these spectral lines are not included in the testing. The disadvantage of the method of the above-mentioned publication exists in the limitation of the statistical testing on the fundamental frequency. However, ASSR are not represented, in the rule, alone by the click-repetition frequency or the fundamental frequency corresponding to the modulation frequency, but they are also represented by one or several harmonic waves for which there is allotted a portion of the response signal power that is not be ignored. An objective verification method limited only to the fundamental wave is therefore not optimal. Methods working in the frequency domain are also described by Stürzebecher and Stürzebecher et al., which use the fundamental wave as well as the relevant harmonic waves for the statistical verification of the ASSR (Stürzebecher E: “Method for hearing screening of newborn by means of steady-state response evoked with high click rate”, European patent application EP 01610060.4; Stürzebecher, E, Cebulla M, Baag M, Thie R: “Verfahren zur objectiven frequenzspezifischen Hörschwellenbestimmung mittels der Amplitude-Modulation Following Response (AMFR)”, European patent application EP1099408 A2). The methods only use either the spectral phase angle of the harmonics for response verification or the spectral phase angle together with the spectral amplitude of the harmonics. A greater sensitivity is achieved for the response verification through the inclusion of the higher harmonics in comparison to methods which work only with the fundamental wave (first harmonic).

With a statistical method working in the time domain, separation of signal and noise is not possible without difficulties as it is described above for the frequency domain. The entire noise power of the asynchronous EEG superimposes the response time function and causes a low sensitivity of response detection. This low sensitivity can also not be compensated with the known statistical test working in the time domain with high test power.

It is the object of the invention to develop a method that makes possible to realize separation of response possible in the frequency domain and the noise caused substantially by the asynchronous EEG in the time domain and to take advantage of the high test power in known statistical test methods in the time domain.

An inventive solution of this object is shown in patent claim 1. Developments of the invention are characterized in the minor claims.

The concept of the invention is the fact that the individual epochs are transformed into the frequency domain. The response is made free of the noise of the asynchronous EEG in the frequency domain. Two possibilities exist for this purpose:

1. The amplitudes of the spectral lines, which contain only noise, are set to zero. The spectral lines of the harmonics remain uninfluenced. The thereby altered spectrum is transformed back into the time domain.

2. All spectral lines, which contain only noise, are eliminated. That means, the spectrum consists only of the harmonics of the response. The thereby altered spectrum is transformed back into the time domain.

A statistical test working in the time domain is used based on the time function of the response obtained after the retransformation into the time domain.

The application of the inventive solution has the following advantages:

First Advantage:

The time function obtained after retransformation from the frequency domain contain essentially only the response. Only small noise portions superimpose the response. Both described methods for “noise filtering” are approximately of equal value; however, the second method has the small advantage that the retransformation requires a somewhat smaller computation effort.

In comparison to the test for original response (not free of noise) in the time domain, the use of a statistical test for a noise-free response in the time domain leads to a higher verification sensitivity. That means, the response is detected more rapidly during hearing screening with a preset stimulus level and time consumption for screening is thereby less. A more accurate threshold determination is possible in an objective threshold determination since the response verification becomes closer to the audible threshold of the patient.

Second Advantage:

An additional important advantage of the described pre-processing of the data in the frequency domain lies in the fact that two response time-functions can be individually depicted in the time domain after pre-processing in the frequency domain and they can be statistically tested individually whereby said two response time-functions are generated through simultaneous auditory stimulation of both ears. This is made possible through different click stimulus rates for the right ear and the left ear.

Additional details, characteristics and advantages of the invention are shown in the following description of an embodiment example.

### EMBODIMENT EXAMPLE

Newborns at the infant ward should be tested by means of ASSR to check whether the hearing ability of both ears is normal. The examination occurs during the natural sleep after feeding. The click stimulus rate for the examination of the right ear is 160 clicks per second, and for the examination of the left ear it is 140 clicks per second. A click stimulus repetition for a duration of 1 second is used in calculations before the start of the examination for each of the two stimulus repetition frequencies and this value is stored in a buffer memory. The two click repetitions for the acoustic stimulation are created through cyclic interrogation of the buffer memory and sent through an earpiece to the right or left ear after D/A conversion at a level of 40 dBnHL. The clock frequency for the D/A conversion is 16,384 per second. The EEG is conducted away from the infant's scalp by means of adhesive electrodes during the acoustic stimulation. The position of electrodes is vertex/neck, ground: forehead. The EEG is amplified and A/D converted. The clock frequency for the D/A converter and the A/D converter has to be synchronized. The clock frequency of the A/D converter is 4096 Hz in the present example. The AID clock frequency is obtained by division (factor 4) of the clock frequency of the D/A converter.

The digitalized EEG is continuously divided into epochs having a length of 1 second. A known device for artifact suppression has the effect that epochs with artifacts are not included in the subsequent evaluations. The artifact-free epochs are transformed into the frequency domain by means of FFT (fast Fourier transformation). The angles and spectral amplitudes of the spectral lines, which correspond to the two fundamental frequencies and the associated harmonic waves, are stored in two data matrixes. Five harmonics are stored for each of the two click stimulus rates. All spectral lines, which contain only EEG noise, are thrown out. Each epoch may contain only integral multiples of the periods of the SSP evoked over the right and left ear to obtain fundamental waves and harmonic waves in the frequency spectrum without sidebands. This is ensured by the selection of the epoch length and the two click repetition frequencies. An FFT of the epochs is possible since the number of scanning values per epoch are an integral number of 2.

The spectra reduced to the 5 harmonics for the responses received for the right and left ear are transformed back into the time domain by means of inverted direct Fourier transformation. Subsequently, one time function exists for the right ear and one for the left ear for each epoch, whereby said time functions are stored in two data matrixes. The statistical testing in the time domain is performed through the Friedman test (Sachs L. Angewandte Statistik. Springer Verlag Berlin, New York, London, 1992). As soon as the first 10 epochs are transformed back, the first test will be conducted whereby the stimulus responses of the right ear and that of the left ear are tested separately. Data acquisition of additional epochs continues simultaneously along with the testing. Each detected epoch is transformed by means of FFT, it is then noise-filtered and transformed back into the time domain. A new test is performed as soon as additional 5 epochs join the first 10 epochs. This sequential testing process is continued until both ASSR (right and left) have been detected, or when only one ASSR is detectable, or when no ASSR is detectable until a maximum number of 120 epochs have occurred. For this example it is assumed that the ASSR of the right ear has been already detected with 25 epochs (25 seconds) and the ASSR of the left ear after 30 epochs (30 seconds). After 25 second, the screening apparatus signals “pass” for the right ear and after an additional 5 seconds it also signals “pass” for the left ear. The simultaneous hearing screening is thus completed for both ears after 30 seconds. In case of a hearing loss on one ear or both ears requiring treatment, the testing time for the signal “fail” is 120 seconds whereby “pass” is signaled at first for the healthy ear after a correspondingly shorter testing period in case of a one-sided hearing impairment. However, the examination continues until the results for both ears are received.

1. A method for objective verification of auditory steady-state responses (ASSR) in the time domain whereby the individual epochs are transformed into the frequency domain.

2. A method according to claim 1, whereby the response is made free from noise of the asynchronous EEG in the frequency domain.

3. A method according to claim 2, whereby the amplitudes of the spectral lines, which contain only noise, are set to zero.

4. A method according to claim 1, whereby the spectral lines of the harmonics remain uninfluenced.

5. A method according to claims 1 through 4, wherein all spectral lines, which contain only noise, are eliminated whereby the spectrum consists now primarily only of the harmonics of the response.

6. A method according to claim 1, wherein all spectral lines, which contain only noise, are eliminated and whereby the spectrum consists primarily of the harmonics of the response only.

7. A method according to claim 6, whereby the spectrum altered in the above manner is transformed back into the time domain.

8. A method according to claim 1 through 7, whereby the time function of the response obtained in the time domain after transformation is used in a statistical test working in the time domain.
